(19) United States
(12) Patent Application Publication
(10) Pub. No.: US 2009/0124017 A1
(43) Pub. Date: May 14, 2009

(54) Sampling Device and Method for the Rapid Detection of Proteins in Mold, Allergens or Other Protein-Containing Substances

(76) Inventors: Brandi M. Cumberland, Pleasanton, CA (US); Jennifer Fung-Sepulveda, Oakland, CA (US); Elias A. Shaheen, Pleasanton, CA (US)

Correspondence Address: THE CLOROX COMPANY, P.O. BOX 24305, OAKLAND, CA, 94623-1305, US

(21) Appl. No.: 12/354,571

(22) Filed: Jan. 15, 2009

Related U.S. Application Data
(62) Division of application No. 11/060,685, filed on Feb. 16, 2005.

(51) Int. Cl. G01N 33/68; B01L 3/00; G01N 21/78

(52) U.S. Cl. 436/86; 422/102; 422/58

(57) ABSTRACT

Provided is sampling device and method for the detection of protein-containing substances. The sampling device includes a first reagent holder coupled to a second reagent holder by an activating member. Coupled to first reagent holder is a first reagent reservoir and coupled to second reagent holder is a second reagent reservoir. The first reagent reservoir is protected from the ambient environment by a first reagent protector and the second reagent reservoir is similarly protected from the ambient environment by a second reagent protector. After a sample has been obtained from the surface of a sampling object, the application of a sufficient activation force on the activating member places the first reagent reservoir in fluid communication with the second reagent reservoir.

### BACKGROUND OF THE INVENTION

1. Field of the Invention

The present invention relates generally to disposable colorimetric sampling devices, and, more specifically, to a disposable colorimetric sampling device for the detection of protein-containing substances.

2. Description of the Related Art

With the increased awareness of health and wellness in the home and other indoor environments, there is growing interest in assessing how efficacious household cleaning products are in denaturing/destroying mold, allergens and other proteins known to potentially cause negative health effects.

Colorimetric assays utilizing sampling devices for the detection of protein in biological samples are commonly used across various industries (biotech, healthcare, food, etc). These sampling devices require minimal manipulation of the protein-containing samples and allow for rapid qualitative and quantitative results. Among the various available colorimetric protein sampling devices is one that utilizes a Bicinchoninic Acid (BCA) protein assay. This assay is based on the initial complexation of Copper [II], hereinafter Cu⁺⁺ or cupric ion, with protein peptides under alkaline conditions, with the reduction to Copper [I], hereinafter Cu⁺ or the cuprous ion, in a concentration-dependent manner. The ligand BCA is then added in excess, and a purple color develops (562 nm peak absorbance) upon binding of BCA with Cu⁺.

Protein detection assays are available through biotechnology companies such as Pierce, Bio-Rad and Pro-tect, a registered product under Biotrace International. In one prior art assay, a plunger, whose reagent-covered swab is used to collect a sample, is inserted into a covered chamber containing reagent and kept separate from the actual plunger.

However, there is a need for the development of a sampling device and method that is equally reliable to the other options already available on the market, but that can also be more conveniently distributed to a larger number of people, more conveniently used in the home, and easily disposed. The current methods of protein detection are unsuitable for home diagnostic applications because of their lack of user-friendly qualities for those not skilled in science, the possibility of misplacing their multiple parts, and the lack of an efficient means of distributing the product to the consumers at a low cost.

Accordingly, there is a need for improved methods and sampling devices for the rapid detection of proteins in mold, allergens or other protein-containing substance for convenient use in a household.

### SUMMARY OF THE INVENTION

The aforementioned needs are satisfied by the sampling device of the present invention, which comprises a first reagent holder coupled to a second reagent holder. Coupled to and disposed within the first reagent holder is a first reagent reservoir. Coupled to and disposed within second reagent holder is a second reagent reservoir.

The first reagent reservoir is protected from the ambient environment by a first reagent protector, which excludes dust and contaminants from contacting the first reagent reservoir. The second reagent reservoir is similarly protected from the ambient environment by a second reagent protector, which excludes dust and contaminants from contacting second reagent reservoir.

The sampling device further includes an activating member that provides a means for placing the first reagent reservoir in fluid communication with the second reagent reservoir.

In one embodiment of the present invention, the first reagent holder is coupled to the second reagent holder by an activating member that is configured as a hinge-like structure. The reagent holders are configured as plate-like structures defining a cavity in which are disposed respective reagent reservoirs. The reagent reservoirs are absorbent non-woven material. The first and second reagent protectors are configured as sheet-like layers overlying their respective reagent reservoirs.

In this embodiment, the activating member allows the first reagent holder to pivot with respect to the second reagent holder and to place the first reagent reservoir in abutting contact and fluid communication with the second reagent reservoir.

In a second embodiment, the sampling device comprises a frangible tube having an opened-end portion and a closed-end portion opposite said closed-end portion. The first reagent holder is the exterior sidewall portion of the tube at its opened-end and the second reagent holder is the interior sidewall portion of the tube. In this embodiment, the first reagent holder is coupled to the second reagent holder by an activating member that is configured as a weakened portion of the tube adjacent its closed-end portion. The tube is frangible at the activating member.

In this second embodiment, the first reagent reservoir is an absorbent non-woven material coupled to the exterior sidewall surface of the tube adjacent its opened-end and the second reagent reservoir is a liquid disposed within the interior of the tube and held in place by capillary force and the second reagent protector.

In this second embodiment, the first protective layer is configured as an envelope surrounding the first reagent reservoir and the second reagent protector comprises liquid silicone, or similar type composition, held in place in the tube below the second reagent reservoir by capillary force before the opened-end portion of the tube is broken off. When the opened-end portion of the tube is broken off at the activating member, the first reagent reservoir and second reagent reservoir are placed in fluid communication.

A method for the fabrication and use of a sampling device for the rapid colorimetric detection of proteins in mold, allergens or other protein-containing substances is described. The method comprises selecting materials of construction for the sampling device that are compatible with a first reagent contained in the first reagent reservoir and a second reagent contained in a second reagent reservoir. Next, the first and second reagent reservoirs are loaded with their respective reagents. Finally, reagent protectors are coupled to their respective reagent holder to protect the reagent form the ambient environment prior to use. The sampling device may be stored until needed.

When needed to perform an analysis, the first reagent protector is removed and the first reagent reservoir is swiped over the surface of a sampling object. The second reagent protector is then removed and the second reagent reservoir is placed in fluid communication with the first reagent reservoir by application of an activation force on the activator member.

A sufficient duration of time is allowed to pass for the development of a positive test result indicator color. If a color develops, the presence of a protein-containing substance on the surface of the test object is confirmed. If no indicator color develops, the absence any protein-containing substance on the surface of the test object is confirmed.

Further features and advantages of the present invention will become apparent to those of ordinary skill in the art in view of the detailed description of exemplary embodiments below, when considered together with the attached drawings and claims.

### BRIEF DESCRIPTION OF THE DRAWINGS

Reference will now be made to the drawings wherein like numerals refer to like parts throughout, and wherein:

FIG. 1 is a top plan view of a sampling device that includes a first reagent holder coupled to a second reagent holder;

FIG. 2 is a cross-sectional view of the sampling device of FIG. 1 taken along the line 2′-2′;

FIG. 3A is a top plan view of the sampling device of FIG. 1 that shows a positive result for the detection of protein-containing substances after a first reagent reservoir has been placed in fluid communication with a second reagent reservoir;

FIG. 3B is a top plan view of the sampling device of FIG. 1 that shows a negative result for the detection of protein-containing substances after a first reagent reservoir has been placed in fluid communication with a second reagent reservoir;

FIG. 4A is a cross-sectional view of the sampling device of FIG. 3A taken along the line 4A′-4A′

FIG. 4B is a cross-sectional view of the sampling device of FIG. 3B taken along the line 4B′-4B′;

FIG. 5 is a cross-sectional view of another embodiment of a sampling device that includes a first reagent holder coupled to second reagent holder.

FIG. 6A a cross-sectional view of the sampling device of FIG. 5 that shows a positive result for the detection of protein-containing substances after a first reagent reservoir has been placed in fluid communication with a second reagent reservoir;

FIG. 6B is a cross-sectional view of the sampling device of FIG. 5 that shows a negative result for the detection of protein-containing substances after a first reagent reservoir has been placed in fluid communication with a second reagent reservoir; and

FIG. 7 is a process flow diagram for a method for the fabrication of the sampling device of FIG. 1 and the use of the sampling device in the rapid calorimetric detection of proteins in mold, allergens or other protein-containing substances; and

FIG. 8 is a process flow diagram for a method for the fabrication of the sampling device of FIG. 5 and the use of the sampling device in the rapid calorimetric detection of proteins in mold, allergens or other protein-containing substances.

### DETAILED DESCRIPTION

The embodiments disclosed herein are described in the context of a sampling device for the rapid detection of proteins in mold, allergens or other protein-containing substances. One of ordinary skill in the art would recognize, however, that the materials and methods disclosed herein will have application in a number of other contexts where sampling and detection of the presence or absence of a particular compound is desirable, particularly where simplicity and ease of use of a sampling/detection device is important.

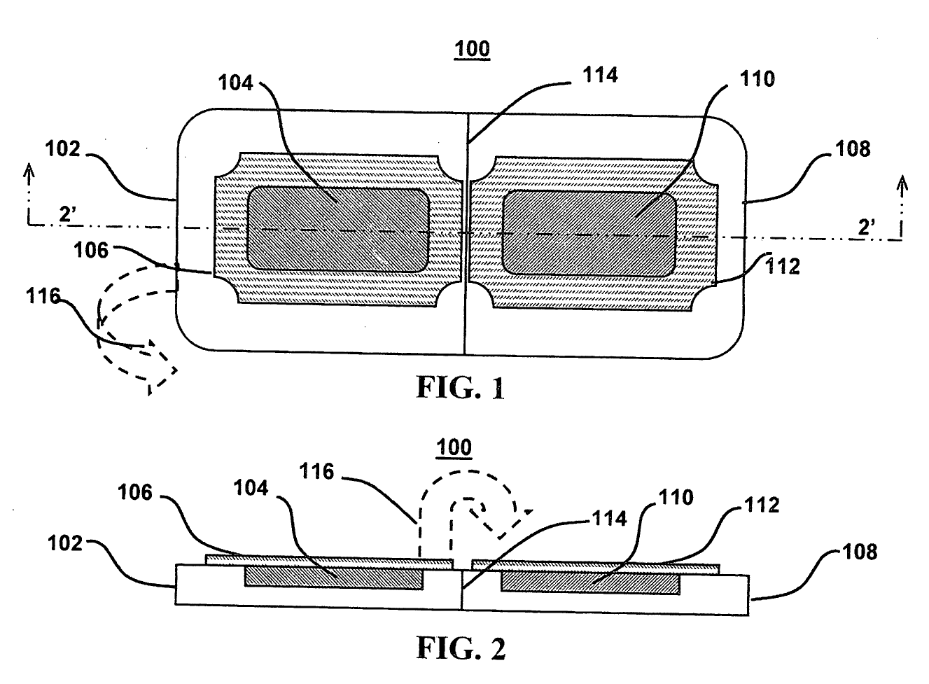

FIG. 1 is a top plan view of a sampling device 100 that includes a first reagent holder 102 coupled to a second reagent holder 108. FIG. 2 is a cross-sectional view of sampling device 100 of FIG. 1 taken along the line 2′-2′. As used herein, positional terms, such as “top” and “bottom” and the like, and directional terms, such as “up” and “down” and the like, are employed for ease of description in conjunction with the drawings. These terms are not meant to indicate that the components of the present invention must have a specific orientation except when specifically set forth below.

Referring to FIGS. 1 and 2 together, in this embodiment, first reagent holder 102 and second reagent holder 108 are similar plate-like members that define cavities, in which first reagent reservoir 104 and second reagent reservoir 110 are, respectively, disposed. In one embodiment, first reagent reservoir 104 and second reagent reservoir 110 comprise absorbent, non-woven materials capable of retaining, for example, reagent liquids, and solid particulates or crystalline compounds.

In one embodiment, first reagent holder 102 is at least partially light transmissive, i.e., transparent or translucent, over at least a portion of reagent holder 102 below first reagent reservoir 104. Second reagent holder 108 is at least partially light transmissive over at least a portion of second reagent holder 108 below second reagent holder 108. Accordingly, first reagent reservoir 104 may be at least partially viewed through the bottom surface of first reagent holder 102 (FIG. 2) and second reagent reservoir 110 may be at least partially viewed through the bottom surface of second reagent holder 108 (FIG. 2).

Overlying first reagent reservoir 104 is a first reagent protector 106; and overlying second reagent reservoir 110 is a second reagent protector 112. In one embodiment, first reagent protector 106 and second reagent protector 112 are flexible sheet-like layers that isolate and protect first reagent reservoir 104 and second reagent reservoir 110, respectively, from dust and other contaminants in the ambient environment. The bottom peripheral edges of first reagent protector 106 and second reagent protector 112 may contain an adhesive material (not shown) to removably couple and provide a seal between reagent protectors 106 and 112 and reagent holders 102 and 108, respectively. Other means to couple reagent protectors 106 and 108 to reagent holders 102 and 108, respectively are possible, such as by way of example and not by way of limitation, static cling.

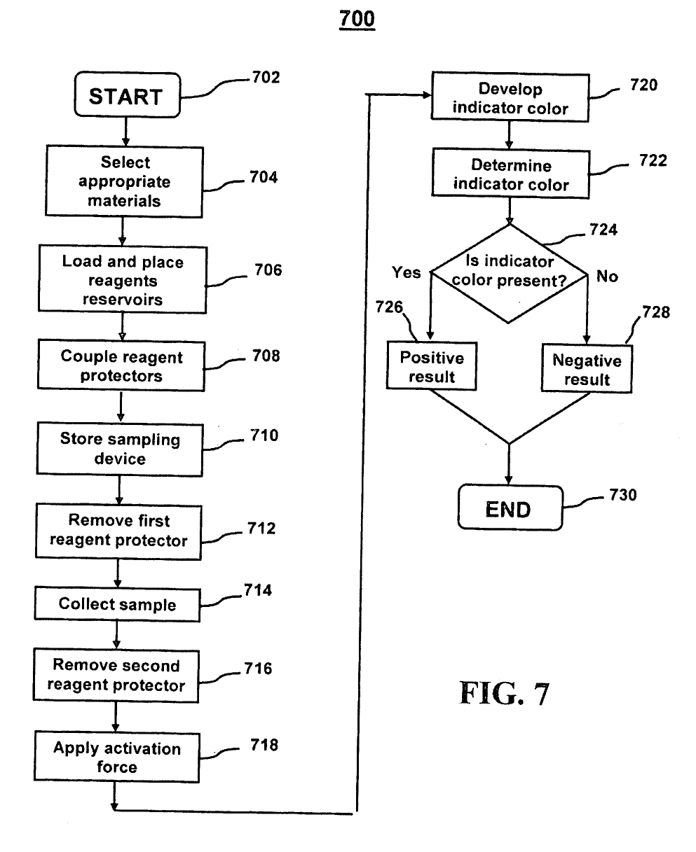

First reagent protector 106 is selected to be compatible with reagent compounds retained in first reagent reservoir 104; and second reagent protector 112 is selected to be compatible with reagent compounds retained in second reagent reservoir 110. First reagent protector 106 and second reagent protector 112 may be transparent, as shown in FIG. 1, wherein first reagent reservoir 104 and second reagent reservoir 110 are visible through first reagent protector 106 and second reagent protector 112, respectively. In other embodiments, first reagent protector 106 and second reagent protector 112 are opaque, whereby first reagent reservoir 104 and second reagent reservoir second reagent reservoir 110, respectively, are protected from light radiation that could affect reagents contained in first reagent reservoir 104 and second reagent reservoir 110. Further, as described more fully below with reference to FIG. 7, first reagent protector 106 and second reagent protector 112 together also isolate first reagent reservoir 104 and second reagent reservoir 110, one from the other, to preclude fluid communication therebetween before use of sampling device 100.

As noted above, first reagent holder 102 is coupled to second reagent holder 108. In the embodiment shown in FIGS. 1 and 2, an activating member 114 couples first reagent holder 102 and second reagent holder 108. In one embodiment, activating member 114 is a hinge-like structure that allows first reagent holder 102 to pivot with respect to second reagent holder 108 by application of an activation force 116 by a user, as indicated in dotted line in FIGS. 1 and 2. First reagent holder 102 and second reagent holder 108 may be formed integrally by, for example, thermoforming or blow-molding from polyethylene or polypropylene. In this embodiment, activating member 114 may be configured as an integrally formed living hinge, well known to those of ordinary skill in the art.

A method of utilizing sampling device 100 for the rapid colorimetric detection of proteins is next described. U.S. Pat. No. 4,839,295 to Smith, incorporated herein by reference in its entirety, discloses a colorimetric Bicinchoninic Acid (BCA) based protein assay. This assay is based on the initial complexation of the cupric ion (Cu⁺⁺) with peptides under alkaline conditions, with the reduction of Cu⁺⁺ to the cuprous ion (Cu⁺) in a concentration-dependent manner (biuret reaction). The ligand BCA is then added in excess, and an intense purple color develops (562 nm peak absorbance) upon binding of BCA with Cu⁺. The intense purple color increases linearly with protein concentration.

FIG. 7 is a process flow diagram for a method 700 for the fabrication of sampling device 100 OF FIG. 1 and the use of sampling device 100 in the rapid colorimetric detection of proteins in mold, allergens or other protein-containing substances. In one embodiment, method 700 utilizes the reagents disclosed by Smith. Referring to FIGS. 1, 2, and 7 together, start operation 702 of method 700 commences the fabrication and use of sampling device 100 for the colorimetric detection of proteins.

Start operation 702 transfers to select appropriate materials operation 704. When it is stated herein that a first operation transfers to a second operation, those of skill in the art understand that the first operation is completed and the second operation is started. In operation 704, the materials of first reagent holder 102, first reagent reservoir 104, first reagent protector 106, second reagent holder 108, second reagent reservoir 110, and second reagent protector 112 are all selected to be compatible with the reagent system disclosed by Smith. For example, first reagent holder 102 and second reagent holder 108 may comprise polyethylene, and first reagent reservoir 104 and second reagent reservoir 110 may comprise terephthalate or polypropylene substrates of absorbent, non-woven materials. When other reagent systems are utilized, the present colorimetric detection method may require other materials of construction for sampling device 100 that are compatible with the reagents of that method. After appropriate materials are selected, operation 704 of method 700 transfers to load and place reagent reservoirs operation 706.

In operation 706, the absorbent non-woven material of first reagent reservoir 104 is loaded. In one embodiment, first reagent reservoir 104 is loaded by being placed in a alkaline solution containing the Cu⁺ ion, such as a copper sulfate (CuSO₄) solution, removed from the solution, and then dried to provide a deposit of CuSO₄ salt on the absorbent non-woven material of first reagent reservoir 104. The absorbent non-woven material of first reagent reservoir 104 is then placed in the cavity defined by first reagent holder 102 (FIG. 2). In addition, in operation 706, the absorbent non-woven material of second reagent reservoir 110 is loaded. In one embodiment, one method of loading includes saturating second reagent reservoir 110 with BCA and placing it in the cavity defined by second reagent holder 108 (FIG. 2). After depositing reagent in first reagent reservoir 104 and second reagent reservoir 110, operation 706 transfers to couple reagent protectors operation 708.

In operation 708, first reagent protector 106 is placed over first reagent reservoir 104 and coupled to first reagent holder 102. In addition, second reagent protector 112 is placed over second reagent reservoir 110 and coupled to second reagent holder 108. Operation 708 provides for protection of first reagent reservoir 104 and second reagent reservoir 110 from dust and other contaminants in the ambient environment that may interfere with the successful operation of method 700. Further, first reagent protector 106 and second reagent protector 112 preclude fluid communication between the reagents contained in first reagent reservoir 104 and second reagent reservoir 110 before commencement of develop indicator color operation 720 described below. After placement of first reagent protector 106 and second reagent protector 112, sampling device 100 may be stored in store sampling device operation 710 until needed to carry out the detection of proteins in mold, allergens or other protein-containing substances.

When sampling device 100 is used to carry out the detection of proteins on an object, operation 710 transfers to remove first reagent protector operation 712. In operation 712, first reagent protector 106 is removed from first reagent holder 102 to expose first reagent reservoir 104 and the reagent, such as Cu⁺⁺ contained therein. As noted above, first reagent protector 106 is in the form of a flexible sheet-like layer. To remove first reagent protector 106 a user peels back and removes the layer to expose first reagent reservoir 104. After first reagent protector 106 is removed from first reagent holder 102, operation 712 transfers to collect sample operation 714.

In operation 714, a user of sampling device 100 wipes the surface of an object for which protein determination is desired with the exposed first reagent reservoir 104. First reagent reservoir 104 of sampling device 100 collects a sample of mold, allergen, etc., which include protein-containing peptides, on the sample object surface and retains the sample on the absorbent non-woven substrate material of first reagent reservoir 104. After the protein sample is secured on first reagent reservoir 104, operation 714 transfers to remove second reagent protector operation 716.

In operation 716, second reagent protector 112 is removed from second reagent holder 102 to expose second reagent reservoir 104 and the reagent, such as BCA, contained therein. After first reagent reservoir 104 is exposed, operation 716 transfers to apply activation force operation 718.

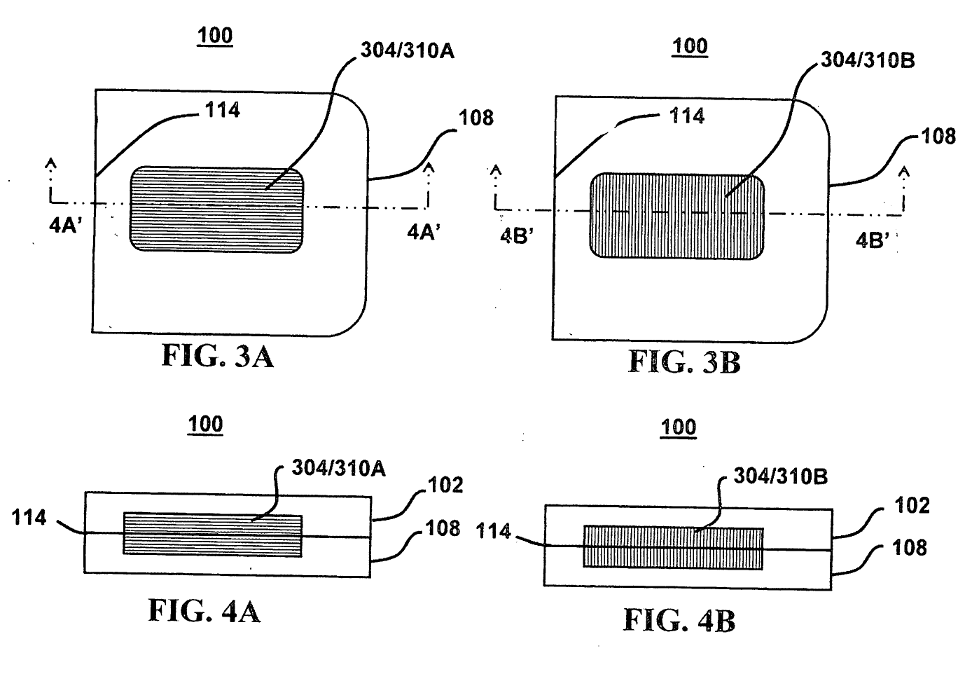

FIG. 3A is a top plan view of sampling device 100 of FIG. 1 that shows a positive result for the detection of protein-containing substances after first reagent reservoir 104 has been placed in fluid communication with second reagent reservoir 110. In FIG. 3A, first reagent holder 102 has been folded over to second reagent holder 108 to place first reagent reservoir 104 in abutting contact and fluid communication with second reagent reservoir 110.

FIG. 3B is a top plan view of sampling device 100 of FIG. 1 that shows a negative result for the detection of protein-containing substances after first reagent reservoir 104 has been placed in fluid communication with second reagent reservoir. In FIG. 3B, first reagent holder 102 has been folded over to second reagent holder 108 to place first reagent reservoir 104 in abutting contact and fluid communication with second reagent reservoir 110. FIG. 4A is a cross-sectional view of sampling device 100 of FIG. 3A taken along the line 4A′-4A′. FIG. 4B is a cross-sectional view of sampling device 100 of FIG. 4B taken along the line 4B′-4B′.

Referring to FIGS. 1, 2, 3A, 3B, 4A, 4B and 7 together, in operation 718 a user of sampling device 100 of FIG. 1 places first reagent reservoir 104 and second reagent reservoir 110 in fluid communication by applying activation force 116 (FIGS. 1 and 2) to fold first reagent holder 102 over, along activating member 114, toward second reagent holder 108. As noted above, activating member 114 is a hinge-like structure, such as a living hinge, that allows first reagent holder 102 to pivot with respect to second reagent holder 108 through application of activation force 116. The pivot action of first reagent holder 102 with respect to second reagent holder 108 allows first reagent reservoir 104 to abuttingly contact and communicate with second reagent reservoir 110. After first reagent holder 102 is pivoted with respect to second reagent holder 108 and first reagent reservoir 104 contacts second reagent reservoir 110, operation 718 transfers to develop indicator color operation 720.

In operation 720 a user of sampling device 100 allows sufficient time for full development of the color that indicates the presence of protein containing substances. For example, in one embodiment an intense purple color develops upon binding of BCA with Cu⁺, as indicated by the horizontal hatching in a reservoir communication positive result 304/310A (FIGS. 3A and 4A). The purple color of a reservoir communication positive result 304/310A (FIGS. 3B and 4B) develops when protein-containing substances, such as mold and allergens, are found on the surface of the object for which the protein determination was made. In operation 720, the color, as indicated by the vertical hatching in a reservoir communication negative result 304/310B, does not develop if protein-containing substances are not found on the surface of the object for which the protein determination was made. Those of ordinary skill in the art will understand that the sensitivity of sampling device 100 and the duration required for full development of reservoir communication positive result 304/310A may be controlled by the strength and nature of the reagents used with sampling device 100. After sufficient time has passed for full development of the indicator color, operation 720 transfers to determine indicator color 722.

In determine indicator color 722, a user of sampling device 100 observes the presence or absence of the above described indicator color. As noted above, first reagent holder 102 and second reagent holder 108 are at least partially light transmissive over at least a portion of the bottom surfaces below their respective reagent reservoirs 104 and 110. Advantageously, a user of sampling device 100 may visually determine the presence of absence of the purple color by looking through first reagent holder 102 folded over on second reagent holder 108 at operation 718. After a user has determined the presence or absence of the indicator color, operation 722 transfers to is indicator color present operation 724.

In operation 724, a user answers the question is the indicator color present at the reservoir communication, i.e., a reservoir communication positive result 304/310A (FIGS. 3A and 4A) or a reservoir communication negative result 304/310B (FIGS. 3B and 4B). If the result of operation 724 is “YES” for the indicator color, operation 724 transfers to positive result operation 726 where the presence of protein-containing substances on the surface of the test object is confirmed resulting in the end of method 700 and end operation 730. If the result of operation 724 is “NO” for the indicator color, operation 724 transfers to negative result operation 728 where the absence of protein-containing substances on the surface of the test object is confirmed resulting in the end of method 700 and end operation 730.

Another embodiment of a sampling device for the detection of protein-containing substances on the surface of a test object is next described. U.S. Pat. No. 5,702,035 to Tsao, incorporated by reference herein in its entirety, discloses a slender tubular container with an opening and closing means for dispensing a liquid such as a medicine or perfume. The container has an applicator opened-end portion wrapped by an absorbing element to allow liquid to flow out while a closed-end portion, opposite the opened-end portion is sealed.

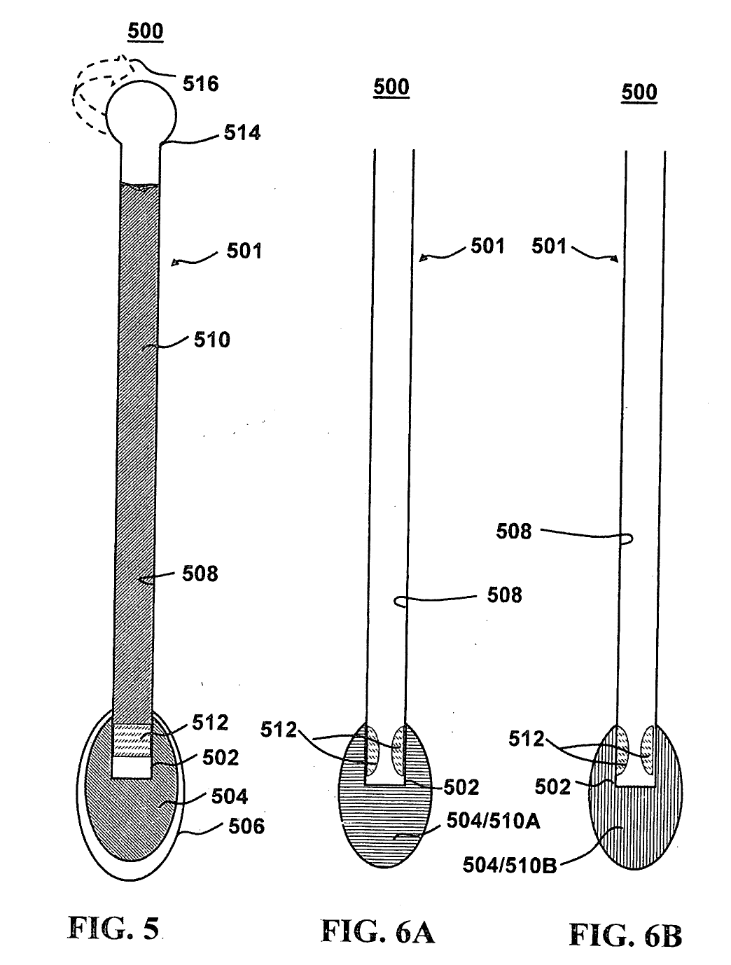

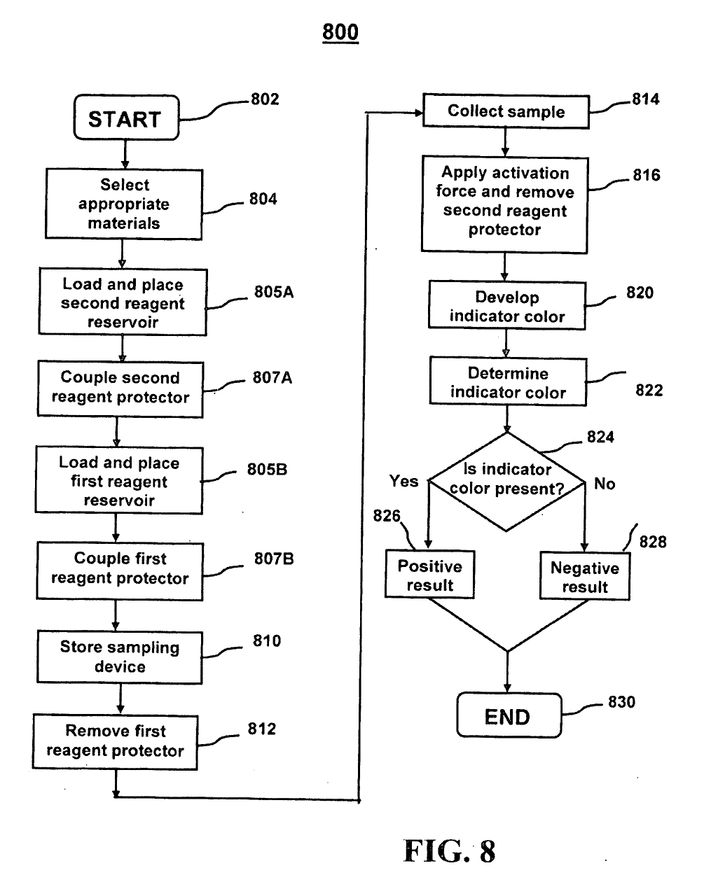

As described more fully below with reference to FIGS. 5 and 8, the opened-end portion of tube 501 is frangible. The containers disclosed are available from “Swab+” of Rancho Cucamunga, Calif., but heretofore, have only been used as dispensing devices for the application of medicines, cosmetics and the like.

FIG. 5 is a cross-sectional view of an embodiment of a sampling device 500 that includes a first reagent holder 502 coupled to a second reagent holder 508. Sampling device 500 is generally configured as a slender tube of the type disclosed by Tsao with an opening and closing means for dispensing a liquid. In one embodiment, first reagent holder 502 is configured as the exterior sidewall surface at an opened-end portion of a tube 501. Further, second reagent holder 508 is configured as the interior sidewall surface of tube 501.

Coupled to first reagent holder 502 is a first reagent reservoir 504. First reagent reservoir 504 comprises an absorbent, non-woven material capable of retaining, for example, reagent liquids, solid particulates, and crystalline compounds. First reagent reservoir 504 is configured as a swab tip coupled to first reagent holder 502 at the exterior sidewall surface tube 501 at its opened-end.

First reagent reservoir 504 is protected from the ambient environment by a first reagent protector 506, which excludes dust and contaminants from contacting first reagent reservoir 504. First reagent protector 506 is configured as an envelope surrounding first reagent reservoir 504. First reagent protector 506 is coupled to first reagent holder 502 above first reagent reservoir 504 at the exterior sidewall surface of tube 501 adjacent its opened-end portion.

Disposed within the interior space defined by tube 501 and coupled to and confined by the tube 501 interior surface of second reagent holder 508, is a second reagent reservoir 510. Second reagent reservoir 510 contains a flowable liquid.

As described in Tsao, second reagent reservoir 510 is protected from the ambient environment by a second reagent protector 512, which excludes dust and contaminants from contacting second reagent reservoir 510. In one embodiment, second reagent protector 512 comprises liquid silicone disposed within the interior space defined by tube 501 and coupled to and confined by the tube 501 interior surface of second reagent holder 508. As is well known to those of ordinary skill in the art, the viscosity of the silicone comprising second reagent protector 512 and the bore of tube 501 may be selected such that the silicone of second reagent protector 512 adheres to the interior surface of tube 501 and remains fixed due to capillary force. Second reagent protector 512 also plugs the flow of second reagent reservoir 510 and precludes fluid communication between first reagent reservoir 504 and second reagent reservoir 510. As silicone is generally inert and immiscible in aqueous solutions, such as BCA, second reagent protector 512 stays in place as an effective means for protecting second reagent reservoir 510 over time.

In the embodiment shown in FIG. 5, an activating member 514 couples first reagent holder 502 and second reagent holder 508. In one embodiment, activating member 514 is a weakened portion of tube 501 adjacent the closed-end portion of tube 501 opposite its opened-end portion. The closed-end portion of tube 501 is frangible at activating member 524, i.e., a user of sampling device 500 may break off and remove the closed-end portion of tube 501 at activating member 514 by application of an activation force 516 indicated in dotted line. As described more fully below with reference to FIG. 8, when the closed-end portion of tube 501 is broken off, second reagent reservoir 510 is put in fluid communication with first reagent reservoir 504. In one embodiment, the absorbent non-woven material of first reagent reservoir 504 is loaded with a deposit of CuSO₄ salt and second reagent reservoir 510 is loaded with liquid BCA. With these reagents, sampling device 500 may be used to detect protein-containing substances such as mold or allergens.

A method of utilizing sampling device 500 for the rapid colorimetric detection of proteins is next described. FIG. 6A is a cross-sectional view of sampling device 500 of FIG. 5 that shows a positive result for the detection of protein-containing substances after second reagent reservoir 510 has been placed in fluid communication with first reagent reservoir 504. FIG. 6B is a cross-sectional view of sampling device 500 of FIG. 5 that shows a negative result for the detection of protein-containing substances after second reagent reservoir 510 has been placed in fluid communication with first reagent reservoir 504. FIG. 8 is a process flow diagram for a method 800 for the fabrication of sampling device 500 and the use of sampling device 500 in the rapid colorimetric detection of proteins in mold, allergens or other protein-containing substances. In one embodiment, method 800 utilizes the reagents disclosed by Smith.

Referring to FIGS. 5, 6A, 6B, and 8 together, start operation 802 of method 800 commences the fabrication and use of sampling device 500 for the calorimetric detection of proteins. Start operation 802 transfers to select appropriate materials operation 804. In operation 804, the materials of first reagent holder 502, first reagent reservoir 504, first reagent protector 506, second reagent holder 508, second reagent reservoir 510, and second reagent protector 512 are all selected to be compatible with the reagent system disclosed by Smith. After appropriate materials are selected, operation 804 of method 800 transfers to load and place second reagent reservoir operation 805A.

In load and place second reagent reservoir operation 805A, second reagent reservoir 510 is loaded and coupled to the interior sidewall of tube 501 making up second reagent holder 508, or more particularly enclosed by tube 501, by merely pouring liquid BCA into the opened-end portion of tube 501 after tube 501 has been placed an inverted configuration with its opened-end portion pointed upwardly, as described in U.S. Pat. No. 5,702,035 to Tsao. After liquid BCA is poured into the opened-end portion of tube 501, operation 805A transfers to transfers to couple second reagent protector operation 807A.

As noted above, second reagent protector 512 comprises liquid silicone, or other inert, immiscible, high-viscosity liquid, disposed within the interior space defined by tube 501 making up second reagent holder 508. Accordingly, in couple second reagent protector operation 807A, second reagent protector 512 is coupled to second reagent holder 508 by merely poring the liquid silicone of second reagent protector 512 over BCA containing second reagent reservoir 510 previously placed in inverted tube 501 during operation 805A. The viscosity of the silicone comprising second reagent protector 512 and the bore of tube 501 may be selected such that the silicone of second reagent protector 512 adheres to the interior surface of tube 501 and remains fixed due to capillary force to plug the flow of second reagent reservoir 510. When tube 501 is placed upright, second reagent reservoir 510 and second reagent protector 512 slide downwardly along tube 501 until the vacuum created at the closed-end portion of tube 501 above the surface of second reagent reservoir 510 ceases further movement. After second reagent protector 512 is coupled to second reagent holder 508, operation 807A transfers to load and place first reagent reservoir operation 805B.

In operation 805B, first reagent reservoir 504 is loaded as described above with reference to method 700 (FIG. 7) for first reagent reservoir 104 (FIG. 1) of sampling device 100. In one embodiment, first reagent reservoir 504 is loaded by being placed in a alkaline solution containing the Cu⁺⁺ ion, such as a copper sulfate (CuSO₄) solution, removed from the solution, and then dried to provide a deposit of CuSO₄ salt on the absorbent non-woven material of first reagent reservoir 104. After first reagent reservoir 504 is loaded with a first reagent, first reagent reservoir 504 is coupled to exterior sidewall of tube 501 making up first reagent holder 502 by means compatible with the reagents and materials of sampling device 500, such as by friction fitting or use of suitable adhesives. After first reagent reservoir is loaded and coupled as described, operation 805B transfers to couple first reagent protector operation 807B.

In couple first reagent protector operation 807B, first reagent protector 506, configured as an envelope, is placed over first reagent reservoir 504 and coupled to first reagent holder 502. First reagent protector 506 may contain an adhesive material (not shown) on the interior upper edge of first reagent protector 506 to removably couple and provide a seal between first reagent protector 506 and the exterior sidewall of tube 501 making up first reagent holder 502. Couple first reagent protector operation 807B and couple second reagent protector operation 807A described above, together provide for protection of first reagent reservoir 504 and second reagent reservoir 510 from dust and other contaminants in the ambient environment that may interfere with the successful operation of method 800. Further, first reagent protector 506 and second reagent protector 512 preclude fluid communication between the reagents contained in first reagent reservoir 504 and second reagent reservoir 510 before commencement of develop indicator color operation 820 described below. After completion of couple first reagent protector operation 807B, store sampling device operation 810 may commence with sampling device 500, as previously described for sampling device 100 (FIG. 1). Sampling device 500 may be stored in store sampling device operation 810 until needed to carry out the detection of proteins in mold, allergens or other protein-containing substances.

When sampling device 500 is used to carry out the detection of proteins on an object, operation 810 transfers to remove first reagent protector operation 812. In operation 812, first reagent protector 506 is removed from first reagent holder 502 to expose first reagent reservoir 504 and the reagent, such as Cu⁺⁺ contained therein. As noted above, first reagent protector 506 is in the form of an envelope coupled to first reagent holder 502 by, for example, an adhesive material. A user peels off first reagent protector 506 a to expose first reagent reservoir 504. After first reagent protector 506 is removed from first reagent holder 102, operation 812 transfers to collect sample operation 814.

In operation 814, a user of sampling device 500 wipes the surface of an object for which protein determination is desired with the exposed first reagent reservoir 504. First reagent reservoir 504 of sampling device 500 collects a sample of mold, allergen, etc., which include protein-containing peptides, on the sample object surface and retains the sample on the absorbent non-woven substrate material of first reagent reservoir 504. After the protein sample is secured on first reagent reservoir 504, operation 814 transfers to apply activation force operation and remove second reagent protector operation 816. In contrast to sampling device 100 (FIG. 1) used in Method 700 (FIG. 7), as shown and describe above, the order of apply activation force operation 718 and remove second reagent protector operation 716 are reversed and combined in Method 800.

In operation 816, while tube 501 is place upright with the closed-end portion of tube 501 above the opened-end portion of 501, a user of sampling device 500 first applies activation force 516 to break off the closed-end portion of tube 501 at activating member 514. When the closed-end portion of tube 501 is broken off, second reagent reservoir 510 and second reagent protector 512 are again subjected to atmospheric pressure. As described in Tsao, since silicone liquid forming second reagent protector 512 is immiscible in BCA, an opening is formed in the center of the tube as the viscous silicone liquid adheres to the internal sidewall of tube 501 (FIGS. 6A and 6B). The BCA contained in second reagent reservoir 510 flows through the opening and comes into fluid communication with first reagent reservoir 504. After activation force 516 is applied to break off the closed-end portion of tube 501 and the BCA contained in second reagent reservoir 510 flows through the opening formed in the silicone liquid of second reagent protector 512, operation 816 transfers to develop indicator color operation 820.

In operation 820, a user of sampling device 500 allows sufficient time for full development of the color that indicates the presence of protein containing substances. For example, in one embodiment an intense purple color develops upon binding of BCA with Cu⁺, as indicated by the horizontal hatching in a reservoir communication positive result 504/310A (FIG. 5A). The purple color of a reservoir communication positive result 504/510A (FIG. 5B) develops when protein-containing substances, such as mold and allergens, are found on the surface of the object for which the protein determination was made. In operation 820, the color on first reagent reservoir 504, as indicated by the vertical hatching in a reservoir communication negative result 504/510B, does not develop if protein-containing substances are not found on the surface of the object for which the protein determination was made. Those of ordinary skill in the art will understand that the sensitivity of sampling device 500 and the duration required for full development of reservoir communication positive result 504/510A may be controlled by the strength and nature of the reagents used with sampling device 500. After sufficient time has passed for full development of the indicator color, operation 820 transfers to determine indicator color 822.

In determine indicator color 822, a user of sampling device 500 observes the presence or absence of the above described indicator color on first reagent reservoir 504. After a user has determined the presence or absence of the indicator color, operation 822 transfers to is indicator color present operation 824.

In operation 824, a user answers the question is the indicator color present at the reservoir communication, i.e., is there a reservoir communication positive result 504/510A (FIG. 5A) or a reservoir communication negative result 5504/510B (FIG. 5B). If the result of operation 824 is “YES” for the indicator color, operation 824 transfers to positive result operation 826 where the presence of protein-containing substances on the surface of the test object is confirmed resulting in the end of method 800 and end operation 830. If the result of operation 824 is “NO” for the indicator color, operation 824 transfers to negative result operation 828 where the absence of protein-containing substances on the surface of the test object is confirmed resulting in the end of method 800 and end operation 830.

The present invention has been described herein in considerable detail to provide those skilled in the art with information relevant to apply the novel principles and to construct and use such specialized components as are required. Specifically, embodiments of the sampling device and method have been described with reference to the detection of protein-containing substance such as mold and allergens. More specifically, the present invention has been described with reference to a calorimeter test. However, it is to be understood that the present invention can be carried out by different equipment, materials and devices, and that various modifications, both as to the equipment and operating procedures, can be accomplished without departing from the scope of the invention itself. Further, the present invention is adaptable to any number of colorimetric tests.

1. A sampling device comprising:
a first reagent holder, configured as a plate-like structure and including a cavity;
a first reagent reservoir coupled to said first reagent holder and disposed within said cavity;
a first reagent protector configured as a sheet-like layer and coupled to said first reagent holder and protecting said first reagent reservoir from the ambient environment;
a second reagent holder, configured as a plate-like structure including a cavity and coupled to said first reagent holder by an activating member;
a second reagent reservoir coupled to said second reagent holder and disposed within said cavity; and
a second reagent protector configured as a sheet-like layer and coupled to said second reagent holder and protecting said second reagent reservoir from the ambient environments;
wherein said activating member is configured as a hinge-like structure;
wherein said first reagent holder is pivotable with respect to said second reagent holder, and wherein said first reagent reservoir and said second reagent reservoir may be placed in abutting contact and fluid communication.

2. The sampling device of claim 1 wherein said first reagent holder is at least partially light transmissive, over at least a portion of said first reagent holder.

3. The sampling device of claim 1 wherein said second reagent holder is at least partially light transmissive, over at least a portion of said second reagent holder.

4. The sampling device of claim 1 wherein said first reagent reservoir and said second reagent reservoir comprise absorbent non-woven material.

5. (canceled)

6. (canceled)

7. (canceled)

8. The sampling device of claim 1:
wherein said first reagent holder and said second reagent holder are integrally formed; and
wherein said activating member comprises a living hinge.

9. (canceled)

10. The sampling device of claim 1 wherein said first reagent protector and said second reagent protector are transparent.

11. The sampling device of claim 1 wherein said first reagent protector and said second reagent protector are opaque.

12. The sampling device of claim 1:
wherein said first reagent protector is removably coupled to said first reagent holder by an adhesive; and
wherein said second reagent protector is removably coupled to said second reagent holder by an adhesive.

13. The sampling device of claim 1:
wherein said first reagent reservoir contains Cu⁺⁺; and
wherein said second reagent reservoir contains BCA.

14. (canceled)

15. (canceled)

16. (canceled)

17. (canceled)

18. (canceled)

19. A sampling device comprising:
a first reagent holder configured as a plate-like member defining a cavity, wherein said first reagent holder is at least partially light transmissive over at least a portion of said first reagent holder;
a first reagent reservoir formed from an absorbent non-woven material and disposed within the cavity defined by said first reagent holder;
a first reagent protector configured as a flexible sheet-like layer, said first reagent protector being coupled to said first reagent holder, wherein said first reagent protector overlies said first reagent reservoir and protects said first reagent reservoir from the ambient environment;
a second reagent holder configured as a plate-like member defining a cavity, said second reagent holder being coupled to said first reagent holder by an activating member configured as a hinge-like structure, and wherein said second reagent holder is at least partially light transmissive over at least a portion of said second reagent holder;
a second reagent reservoir formed from an absorbent non-woven material and disposed within the cavity defined by said second reagent holder; and
a second reagent protector configured as a flexible sheet-like layer, said second reagent protector being coupled to said second reagent holder, wherein said second reagent protector overlies said second reagent reservoir and protects said second reagent reservoir from the ambient environment;
wherein said first reagent holder is pivotable with respect to said second reagent holder, and wherein said first reagent reservoir and said second reagent reservoir may be placed in abutting contact and fluid communication.

20. The sampling device of claim 19:
wherein said first reagent holder and said second reagent holder are integrally formed; and
wherein said activating member comprises a living hinge.

21. The sampling device of claim 19 wherein said first reagent protector and said second reagent protector are transparent.

22. The sampling device of claim 19 wherein said first reagent protector and said second reagent protector are opaque.

23. The sampling device of claim 19:
wherein said first reagent protector is removably coupled to said first reagent holder by an adhesive; and
wherein said second reagent protector is removably coupled to said second reagent holder by an adhesive.

24. The sampling device of claim 19:
wherein said first reagent reservoir contains Cu⁺⁺; and
wherein said second reagent reservoir contains BCA.

25. The sampling device of claim 19 wherein an indicator color develops when said first reagent reservoir and said second reagent reservoir are placed in fluid communication, and a protein-containing compound is detected.

26. The sampling device of claim 24 wherein a purple color develops when said first reagent reservoir and said second reagent reservoir are placed in fluid communication, and a protein-containing compound is determined.

27. (canceled)

28. (canceled)

29. (canceled)

30. (canceled)

31. (canceled)

32. A method for the fabrication and use of a sampling device for the rapid calorimetric detection of proteins in mold, allergens or other protein-containing substances comprising:
preparing a sampling device according to the device of claim 1;
selecting materials of construction of said sampling device that are compatible with said first reagent and said second reagent;
loading said first reagent reservoir with said first reagent;
loading said second reagent reservoir with said second reagent;
coupling said first reagent protector to said first reagent holder, said first reagent protector overlying said first reagent reservoir;
coupling said second reagent protector to said second reagent holder, said second reagent protector overlying said second reagent reservoir;
storing said sampling device until use;
removing said first reagent protector from said first reagent holder;
collecting a sample from the surface of a sampling object;
removing said second reagent protector from said second reagent holder;
applying an activation force on said activating member to place said first reagent reservoir in fluid communication with said second regent reservoir;
developing an indicator color; and
determining the presence or absence of said indicator color.

33. (canceled)

34. (canceled)

35. The method of claim 32:
wherein said first reagent reservoir contains Cu⁺⁺; and
wherein said second reagent reservoir contains BCA.

36. (canceled)
